(19) United States
(12) Patent Application Publication
(10) Pub. No.: US 2009/0209988 A1
(43) Pub. Date: Aug. 20, 2009

(54) PATIENT CONFIGURED DEVICE, A KIT AND A METHOD FOR TREATMENT OF DISORDERS IN THE HEART RHYTHM REGULATION SYSTEM

(75) Inventors: Jan Otto Solem, Stetten (CH); Stevan Nielsen, Rottenburg Am Neckar (DE); Ib Joergensen, Haigerloch (DE); Gerd Seibold, Ammerbuch (DE); Bodo Quint, Rottenburg (DE)

Correspondence Address: THE NATH LAW GROUP, 112 South West Street, Alexandria, VA, 22314, US

(73) Assignee: Syntach AG, Schaffhausen (CH)

(21) Appl. No.: 12/227,348

(22) PCT Filed: May 17, 2006
(86) PCT No.: PCT/EP2006/062401

§ 371 Date: Nov. 14, 2008

(51) Int. Cl. A61B 17/32; G06F 17/00

(52) U.S. Cl. 606/167; 700/98; 700/119

(57) ABSTRACT

A patient-configured tissue cutting device is disclosed, which is structured and arranged to be inserted through the vascular system into a body vessel adjacent to the heart and/or into the heart, and to be subsequently subjected to a change of shape in order to penetrate into the heart tissue. The patient configured tissue cutting device may thus advantageously be used for treating disorders to the heart rhythm regulation system of a specific patient. A kit of devices provides a plurality of devices for creating a lesion pattern for treating such disorders, a device, system, and method for determining the shape of said vessel, are also disclosed.

### FIELD OF THE INVENTION

The present invention relates to treatment of disorders in the heart rhythm regulation system and, specifically, to a tissue cutting device, a kit of shape-changing devices, a method for treating such disorders, method of manufacture of said device, a vessel shape determining device and method of use thereof, and a system.

### BACKGROUND OF THE INVENTION

The circulation of blood in the body is controlled by the pumping action of the heart. The heart expands and contracts by the force of the heart muscle under impulses from the heart rhythm regulation system. The heart rhythm regulation system transfers an electrical signal for activating the heart muscle cells.

The normal conduction of electrical impulses through the heart starts in the sinoatrial node, travels across the right atrium, the atrioventricular node, the bundles of His and thereafter spread across the ventricular muscle mass. Eventually when the signal reaches the myocytes specialized in only contraction, the muscle cell will contract and create the pumping function of the heart (see FIG. 1).

The electrical impulses are transferred by specially adapted cells. Such a cell will create and discharge a potential over the cell membrane by pumping ions in and out of the cell. Adjacent cells are joined end-to-end by intercalated disks. These disks are cell membranes with a very low electrical impedance. An activation of a potential in a cell will propagate to adjacent cells thanks to the low impedance of the intercalated disks between the cells. While being at the embryonic stage, all heart muscle cells, the myocytes, have the ability to create and transfer electrical signals. During evolution the myocytes specialize and only those cells necessary for maintaining a stable heart-rate are keeping the ability to create and send electrical impulses. For a more thorough explanation of the propagation of electrical signals in the heart, see e.g. Sandöe, E. and Sigurd, B., Arrhythmia, Diagnosis and Management, A Clinical Electrocardiographic Guide, Fachmed AG, 1984.

The heart function will be impaired if there is a disturbance on the normal conduction of the electrical impulses. Atrial fibrillation (AF) is a condition of electrical disorder in the heart rhythm regulation system. In this condition, premature and fast signals irregularly initiating muscle contractions in the atria as well as in the ventricles will be started in ectopic sites, that is areas outside the sinoatrial node. These signals will be transmitted erratically all over the heart. When more than one such ectopic site starts to transmit, the situation becomes totally chaotic, in contrast to the perfect regularity in a healthy heart, where the rhythm is controlled from the sinoatrial node.

Atrial fibrillation is a very common disorder, thus 5% of all patients that undergo heart surgery suffer from AF. 0.4-2% of a population will suffer from AF, whereas 10% of the population over the age of 65 suffers from AF. 160 000 new cases occur every year in the US and the number of cases at present in the US is estimated to be around 3 million persons. Thus, treatment of atrial fibrillation is an important topic.

Typical sites for ectopic premature signals in AF may be anywhere in the atria, in the pulmonary veins (PV), in the coronary sinus (CS), in the superior vena cava (SVC) or in the inferior vena cava (IVC). There are myocardial muscle sleeves present around the orifices and inside the SVC, IVC, CS and the PVs. Especially around the orifice of the left superior pulmonary vein (LSPV) such ectopic sites are frequent, as well as at the orifice of the right superior pulmonary vein (RSPV). In AF multiple small circles of a transmitted electrical signal started in an ectopic site may develop, creating re-entry of the signal in circles and the circle areas will sustain themselves for long time. There may be only one ectopic site sending out signals leading to atrial flutter, or there may be multiple sites of excitation resulting in atrial fibrillation. The conditions may be chronic or continuous since they never stop. In other cases there may be periods of normal regular sinus rhythm between arrhythmias. The condition will then be described as intermittent.

In the chronic or continuous cases, the atrial musculature undergoes an electrical remodelling so that the re-entrant circuits sustain themselves continuously. The patient will feel discomfort by the irregular heart rate, sometimes in form of cannon waves of blood being pushed backwards in the venous system, when the atria contract against a closed arterio-ventricle valve. The irregular action of the atria creates standstill of blood in certain areas of the heart, predominantly in the auricles of the left and right atrium. Here, blood clots may develop. Such blood clots may in the left side of the heart get loose and be taken by the blood stream to the brain, where it creates disastrous damage in form of cerebral stroke. AF is considered to be a major cause of stroke, which is one of the biggest medical problems today.

Today, there are a few methods of treating the problems of disorders to the heart rhythm regulation system. Numerous drugs have been developed to treat AF, but the use of drugs is not effective to a large part of the patients. Thus, there has also been developed a number of surgical therapies.

Surgical therapy was introduced by Drs. Cox, Boineau and others in the late 1980s. The principle for surgical treatment is to cut all the way through the atrial wall by means of knife and scissors and create a total separation of the tissue. Subsequently the tissues are sewn together again to heal by fibrous tissue, which does not have the ability to transmit myocardial electrical signals. A pattern of cutting was created to prohibit the propagation of impulses and thereby isolate the ectopic sites, and thus maintain the heart in sinus rhythm. The rationale for this treatment is understandable from the description above, explaining that there must be a physical contact from myocyte to myocyte for a transfer of information between them. By making a complete division of tissue, a replacement by non-conductive tissue will prohibit further ectopic sites to take over the stimulation. The ectopic sites will thus be isolated and the impulses started in the ectopic sites will therefore not propagate to other parts of the heart.

It is necessary to literally cut the atria and the SVC and the IVC in strips. When the strips are sewn together they will give the impression of a labyrinth guiding the impulse from the sinoatrial node to the atrioventricular node, and the operation was consequently given the name Maze. The cutting pattern is illustrated in FIG. 2 and was originally presented in J L Cox, T E Canavan, R B Schuessler, M E Cain, B D Lindsay, C Stone, P K Smith, P B Corr, and J P Boineau, The surgical treatment of atrial fibrillation. II. Intraoperative electrophysiologic mapping and description of the electrophysiologic basis of atrial flutter and atrial fibrillation, J Thorac Cardiovasc Surg, 1991 101: 406-426. The operation has a long-time success of curing patients from AF in 90% of the patients. However, the Maze operation implicate that many suture lines have to be made and requires that the cuts are completely sealed, which is a demanding task for every surgeon that tries the method. The operation is time consuming, especially the time when the patients own circulation has to be stopped and replaced by extracorporeal circulation by means of a heart-lung machine. Thus mortality has been high and the really good results remained in the hands of a few very trained and gifted surgeons.

The original Maze operation has therefore been simplified by eliminating the number of incisions to a minimum, still resulting in a good result in most cases. The currently most commonly used pattern of incisions is called Maze III (see FIG. 3).

Other methods of isolating the ectopic sites have also been developed recently. In these methods, the actual cutting and sewing of tissue has been replaced by methods for killing myocyte cells. Thus, one may avoid separating the tissue, instead one destroy the tissue by means of heat or cooling in the Maze pattern to create a lesion through the heart wall. The damaged myocyte tissue can not transfer signals any more and therefore the same result may be achieved. Still the chest has to be opened, and the heart stopped and opened. Further, the energy source has to be carefully controlled to affect only tissue that is to be destroyed.

A large number of devices have now been developed using various energy sources for destroying the myocyte tissue. Such devices may use high radio frequency energy, as disclosed in e.g. U.S. Pat. No. 5,938,660, or microwaves, ultrasound or laser energy. Recently, devices have been developed for catheter-based delivery of high radio frequency energy through the venous and or arterial systems. However, this has so far had limited success due to difficulties in navigation and application of energy and also late PV stenosis has been reported. Further, devices using cooling of tissue has used expanding argon gas or helium gas to create temperatures of −160° C. Using an instrument with a tip, tissue can be frozen and destroyed.

The devices according to prior art are accompanied with problems, such as the inability to prevent cutting action, and thus the inability to regulate this cutting action in such way that a perhaps sensitive tissue surrounding the tissue to be cut is protected from cutting. It may be of interest to prevent the cutting action to proceed further than the actual outer contour of the tissue to be cut. Furthermore, the devices according to the prior art are naturally incapable of regulating and assuring that a homogenous cutting action, i.e. wherein the tissue is cut in at substantially the same rate and simultaneously, of the tissue to be cut is performed.

Also, prior art is silent about a cutting device, which performs homogenous cutting action, whereby the cutting action to be cut is cut in substantially the same speed, or which cutting device is easier to fixate in the desired cutting position, to ensure that the cutting action is performed in a predicted and/or regulated manner. Hence, there is a need for an improved tissue device and method that provides a more advantageous way of cutting action, and in particular allowing for increased flexibility, cost-effectiveness of patient treatment, or patient safety.

### SUMMARY OF THE INVENTION

Accordingly, the present invention seeks to mitigate, alleviate or eliminate one or more of the above-identified deficiencies and to provide a new device, kit of devices, method of manufacture of said device, a vessel shape determining device and method of use thereof, and a system, suitable for treatment of disorders to the heart rhythm regulation system of the kinds referred to, according to the appended independent claims.

For this purpose a tissue cutting device according to claim 1 is provided, wherein the device is structured and arranged to be inserted in a temporary delivery shape through the vascular system into a body vessel adjacent to the heart and/or into the heart and to be subsequently subjected to a change of shape, from said temporary delivery shape via an expanded delivered shape to a further expanded shape, extending at least beyond an inner surface of said tissue, in order to create cutting action configured for cutting said heart tissue and/or said body vessel, and wherein the cutting device has a shape adapted to the actual shape of said body vessel adjacent to the heart and/or said heart.

Advantageous features of the invention are defined in the dependent claims.

### BRIEF DESCRIPTION OF THE DRAWINGS

The invention will now be described in further detail by way of example under reference to the accompanying drawings, on which:

FIG. 1 is a schematic view of the transmission of electrical signals in the heart;

FIG. 2 is a schematic view of a pattern of cutting tissue of the heart wall according to the Maze-procedure for treating disorders to the heart rhythm regulation system;

FIG. 3 is a schematic view of a simplified pattern according to the Maze III-procedure, wherein the heart is seen from behind;

FIGS. 4a-4c are perspective schematic views of a tissue cutting device according to an embodiment of the invention, wherein FIG. 4a shows the tissue cutting device in a first, temporary shape, FIG. 4b shows the tissue cutting device in a second, permanent shape, and FIG. 4c illustrates the tissue cutting device having sharp edges;

FIG. 5 show different embodiment of the tissue cutting device,

FIGS. 6-8 illustrate three different embodiments of accessing the vascular system;

FIG. 9 illustrates a guide wire being inserted into the coronary sinus;

FIG. 10 illustrates a guide wire being inserted into the coronary sinus and a guide catheter being inserted with its tip at the orifice of the coronary sinus;

FIG. 11 is a view similar to FIG. 10 showing a first tissue cutting device being inserted into the coronary sinus;

FIGS. 12 and 13 illustrate a guide wire having been inserted into the left atrium;

FIGS. 14-16 illustrate the carrying and deployment of a tissue cutting device by means of a delivery catheter;

FIGS. 17-19 illustrate the deployment of a tissue cutting device in the left superior pulmonary vein;

FIGS. 20-23 illustrate the insertion of a tissue cutting device into the inferior and superior vena cava;

FIG. 24 illustrate the deployment of a tissue cutting device according to FIG. 5 in the left atrium;

FIG. 25 illustrate the deployment of a tissue cutting device according to FIG. 5 in the right atrium; and

FIG. 26 illustrate a tissue lesion creating cutting device according to FIG. 5a located in the left atrium.

### DETAILED DESCRIPTION OF EMBODIMENTS

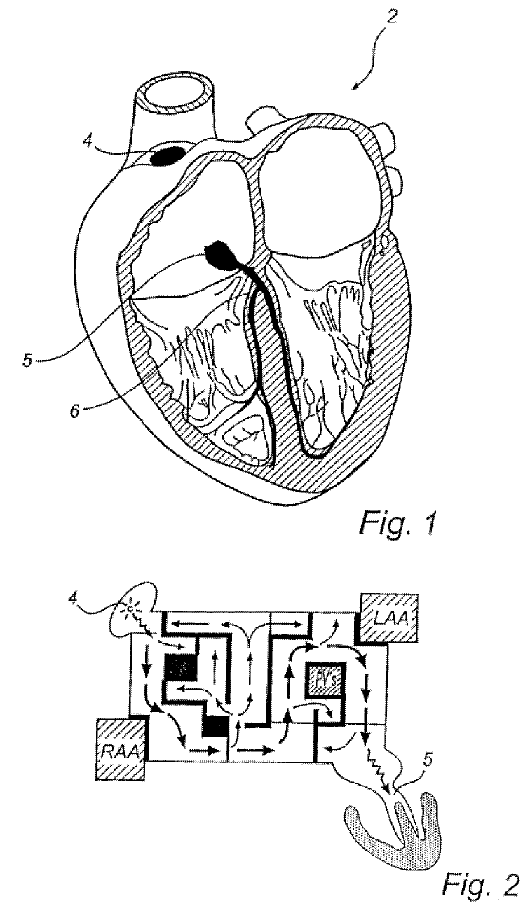

Referring now to FIGS. 1-3, the problems of disorders to the heart rhythm regulation system and the leading current method of treating these problems will be described. In FIG. 1, a heart 2 is shown and the controlling of the heart rhythm is indicated. The heart rhythm is normally controlled from the sinoatrial node 4. The sinoatrial node 4 transmits electrical signals, which are propagated through the heart wall by means of special cells forming an electrical pathway. The electrical signals following the electrical pathway will coordinate the heart muscle cells for almost simultaneous and coordinated contraction of the cells in a heart atrium and heart ventricle. The normal conduction of electrical impulses through the heart starts in the sinoatrial node 4, travels across the right atrium, the atrioventricular node 5, the bundles of His 6 and thereafter spread across the ventricular muscle mass. In a disordered situation, electrical signals are started in heart cells outside the sinoatrial node 4, in so called ectopic sites. These electrical signals will disturb the coordination of the heart muscle cells. If several ectopic sites are present, the signal transmission becomes chaotic. This will be the cause of arrhythmic diseases, such as atrial fibrillation and atrial flutter.

An existing method for treating these diseases is based on isolating the ectopic sites in order to prevent the electrical signals started in these ectopic sites to propagate in the heart wall. Thus, the heart wall is cut completely through for interrupting the coupling between cells that transmit erratic electrical signals. The thus created lesion will be healed with fibrous tissue, which is unable to transmit electrical signals. Thus, the path of the electrical signals is blocked by this lesion. However, since the location of the ectopic sites may not always be known and may be difficult to determine or since there might be multiple ectopic sites, a special cutting pattern has been developed, which will effectively isolate ectopic sites. Thus, the same pattern may always be used regardless of the specific locations of the ectopic sites in each individual case. The procedure is called the “Maze”-procedure in view of the complicated cutting pattern. In FIG. 2, the Maze-pattern is illustrated.

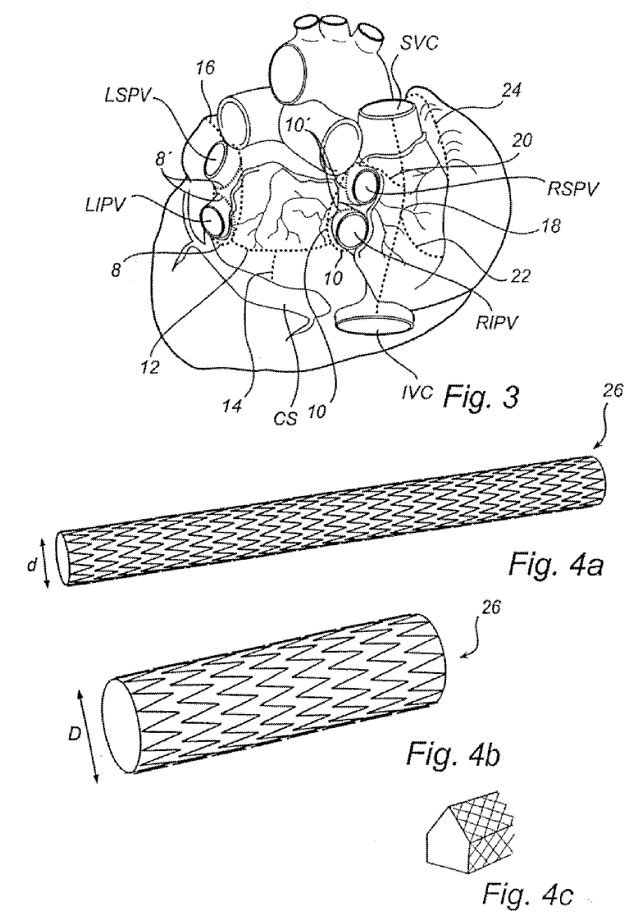

However, as is evident from FIG. 2, the cutting pattern is extensive and complex and requires a difficult surgery. Thus, the Maze-pattern has been evolved in order to minimize the required cuttings and simplify the pattern as much as possible. Currently, a Maze III-pattern is used, as shown in FIG. 3. This pattern is not as complicated, but would still effectively isolate the ectopic sites in most cases. The Maze III-pattern comprises a cut 8 around the left superior pulmonary vein (LSPV) and the left inferior pulmonary vein (LIPV) and a corresponding cut 10 around the right superior pulmonary vein (RSPV) and the right inferior pulmonary vein (RSPV); a cut 12 connecting the two cuts 8 and 10 around the pulmonary veins (PV); a cut 14 from this connecting cut to the coronary sinus (CS); a cut 16 from the left PVs to the left atrial appendage; a cut 18 from the inferior vena cava (IVC) to the superior vena cava (SVC); a cut 20 connecting the cut 10 around the right PVs and the cut 18 between the IVC and the SVC; a cut 22 from the cut 18 between the IVC and the SVC along the right lateral atrium wall; and a cut 24 isolating the right atrial appendage. Thus, a pattern, which is less complex and which effectively isolates the ectopic sites, has been established. In some cases, all cuts may not be needed. For example, the occurrence of ectopic sites often starts around the orifices of the PVs and, therefore, it may be sufficient to make the cuts 8, 10 around the PVs. Further, as indicated with the lines 8′ and 10′, the cuts around the PVs may be done along each PV orifice instead of in pairs.

According to the patient specific tissue cutting devices, there is provided an advantageous possibility of cutting through the heart wall. Thus, a patient specific, and similar pattern to the Maze III-pattern should also be achieved according to this new manner. However, as mentioned above, it may not in all cases be required that all cuts of the Maze III-pattern are made. Furthermore, some cuts may be preferably interrupted after some time of cutting, to not cut tissue in the vicinity of the tissue to be cut, or if only a part of the tissue is to be cut, for example if the ectopic site to be isolated is located close to surface first subjected to cutting. It may also be possible to activate a cutting action after some time of subjection to a stimuli by providing said cutting device which

An already filed non-published international application, of same applicant as the present application, with application number PCT/EP2005/005363, a heart wall tissue lesion creating cutting device is described and the new manner of performing the cuts through the heart wall is explained, which international application hereby is integrated herein in its entirety.

This heart wall tissue lesion creating cutting device 26 (hereinafter called cutting device) is shown in FIG. 4a in a first state, in which the cutting device 26 is tubular and has a first diameter d. The cutting device 26 is shown in FIG. 4b in a second state, in which the cutting device 26 is tubular and has a second diameter D, which is larger than the first diameter d. The cutting device 26 is formed of a shape memory material, which has the ability of memorizing a permanent shape that may significantly differ from a temporary shape. The shape memory material will transfer from its temporary to its memorized, permanent shape as a response to a suitable stimulus. The stimulus may be exposure to a raised temperature, such as a temperature above e.g. 30° C. that may be caused by the body temperature. The stimulus may suitably be combined with the release of a restraining means, which may keep the shape memory material from assuming its permanent shape.

In an embodiment of the present invention such a cutting device is structured and arranged to be inserted in a temporary delivery shape through the vascular system into a body vessel adjacent to the heart and/or into the heart. Thereafter, the cutting device is subjected to a change of shape, from said temporary delivery shape via an expanded delivered shape to a further expanded shape, extending at least beyond an inner surface of said tissue. Thereby, a cutting action configured for cutting said heart tissue and/or said body vessel is obtained. The cutting device has a shape that is adapted to the actual shape of said body vessel adjacent to the heart and/or said heart. This may for example be a shape that is substantially corresponding to said body vessel adjacent to the heart and/or said heart. In this way one may ensure that homogenous cutting action, i.e. that the tissue is cut in at substantially the same rate and simultaneously, is performed in the tissue intended to be cut. This may for example be demanded if a sensitive tissue surrounds one part of the tissue to be cut. For example, if the cutting device is not shaped in accordance with said body vessel adjacent to the heart and/or said heart, one part of the cutting device may travel through the tissue to be cut and further into adjacent tissue while one part of the cutting device perhaps only just has initiated cutting action or not initiated cutting action at all. A cutting device, which is shaped in accordance with the tissue to be cut, will for instance perform cutting action in substantially the same speed in all directions. Hereby, homogenous cutting action is ensured. Another benefit with the shaped cutting device is that it will be much easier to fixate the cutting device, since it fits in the tissue to be cut. Hereby, the cutting action will be more exactly predicted and regulated.

Hereinafter, it is elucidated in more detail how the shaping of the cutting device may be performed. In an embodiment, a template/measure of the actual patient tissue is firstly produced by means of an image of the tissue, such as heart, atrium, ventricle, or blood vessel adjacent the heart. The image may be taken by means of a suitable imaging modality. The produced images should have a sufficient resolution to ensure that the anatomical structure of the patient tissue is reproducible from the image. The image may be a three-dimensional (3-D) image acquired by known methods of creating images of anatomic structures, such as Magnetic Resonance Tomography (MRT), Magnetic Resonance Imaging (MRI), Positron Emission Tomography (PET), CT or CAT scans, or Ultrasonic imaging systems. Since it is possible to provide momentaneous pictures with these methods, the difficulty of picturing tissue that is constantly moving, such as the heart tissue, is overcome. In this way, for instance, a 3-D image of the heart muscle and surrounding vessels may be provided, including information concerning the interior and exterior shape of the heart muscle structure or vessels, as well as their thickness and eventually distribution of tissue types along the muscle or vessel tissue. Also, the heart cycle movement is capturable my means of a series of such images, providing a dynamic measure for adapting tissue cutting devices to specific patient anatomy.

Cardiac ultrasonic imaging may for instance be performed intracardially by introducing the ultrasonic measuring head into the body. The measuring head may for this purpose be introduced with a catheter delivery system into the heart or vessel system thereof. Alternatively, a device introduced through the mouth and oesophagus of a patient may be positioned closer to the heart than from the exterior of the body. Thus, image quality, resolution or frame frequency of captured heart cycle motion may be improved.

Different suitable imaging systems and modalities exist today in respect of creating images of anatomic structures, such as Magnetic Resonance Tomography (MRT), Magnetic Resonance Imaging (MRI), Positron Emission Tomography (PET), CT or CAT scans, and Ultrasonic imaging systems. These are examples of imaging methods useful to achieve the beneficial characteristics of the present invention. Other medical imaging methods and modalities may provide even more advantageous image information.

In more detail, Magnetic Resonance Tomography (MRT) is a method in which a magnetic camera alters the magnetization of hydrogen nuclei in the body, by the aid of a combination of magnetic field and radio waves (RF-pulses). After each RF-pulse the atomic nuclei return to their original magnetization, simultaneously as emitting radio waves. The radio waves are caught by an antenna, and a computer transforms the information into a series of cross-sectional images, i.e. Magnetic Resonance Imaging (MRI). A magnetic camera may be used to obtain anatomic pictures, such as abdomen, vessels, etc., but may also be used to study a bodily function, such as the metabolism. MRI modalities may be used for cardiac inventigations.

Positron Emission Tomography (PET) uses the fact that radioactive isotopes disintegrate by emitting positrons (positive electrons). When the positron have spent its kinetic energy and encounter an electron, the positron will annihilate, i.e. the rest mass is transformed into photons. These photons are sent out in 180 degrees with respect of each other, and both are detected in opposite detectors. Their simultaneous origin is used to develop a time frame at the first detection. If another detection is obtained within a certain time limit, such as approximately 10 ns, these events are matched together to a coincidence pair. On the distance between these events a disintegration has occurred. Coincidence pairs are collected around the object to be imaged, and through reconstruction technique recreate a three dimensional image of the radioactive distribution in the object to be imaged, e.g. the cardiac area in a patient body.

CT (Computer Tomography) scans are special x-ray tests that produce cross-sectional images of the body using x-rays and a computer. 3D CT images are based on “pictures” of slices of the body. CT scans are frequently used to evaluate the brain, neck, spine, chest, abdomen, pelvis, and sinuses. CT has become a commonly performed procedure. Scanners are found not only in hospital x-ray departments, but also in outpatient offices. CT has revolutionized medicine because it allows doctors to see diseases that, in the past, could often only be found at surgery or at autopsy. CT is noninvasive, safe, and well-tolerated. It provides a highly detailed look at many different parts of the body, such as the cardiac area.

Ultrasonic imaging systems use sharply focused sound beams to produce pictures similar to X-rays that show an object's internal structure. To do this, one or more transducers scan over the object, taking reflection or transmission data at many points and assembling the information into an image. Changes in echo position or amplitude will correspond to changes in the body part under investigation. By mapping these changes it is possible to generate a very detailed image, e.g. of the cardiac area.

It is also possible to provide a catheter with members, such as wires, extending in a three dimensional pattern, which members are in communication with a computer. Thus, it will be possible to insert the catheter, in a known way, into the tissue to be imaged, such as the heart. Thereafter, the members extending in a three dimensional pattern are released inside for example the heart or blood vessel whereby they will strive expandingly, until they encounter the wall of the tissue, such as the heart or vessel wall. The members extending an a three dimensional manner will then interact with the walls of said vessel, and thereby create a certain formation in space. The formation of these members extending an a three dimensional manner are then communicated to the computer, which computer thereafter calculates the shape of the tissue, such as the heart or vessel.

When the template of the image of the tissue to be cut is obtained, the cutting device may be shaped accordingly. In one embodiment a copy of the tissue to be cut is made in a suitable material, such as plaster. Alternatively, a technology known as Rapid Manufacturing (RM) may be used to shorten the design and production cycle of the template. For instance layer manufacturing or Solid Freeform Fabrication (SFF) may be used for this purpose, in which the arbitrary shape of the patient anatomy, based on the medical image, is be produced in a single process by adding successive layers of material. RM may also provide the fast fabrication of the tools required for mass production, such as specially-shaped molds, dies, and jigs. The application of layer manufacturing to make the components used in production is termed Rapid Tooling (RT). It may be applied to injection molding, investment casting, and mold casting processes. In this way, a patient specific device may be reproduced if it shows to be suitable for a wide range of patients.

The copy or template of the patient specific tissue has a somewhat larger dimension than the actual tissue to be cut. Thereby, the cutting device may be formed into its memory shape, which preferably has a larger dimension than the actual image, to thereby ensure that the cutting device will cut through the said tissue when the cutting device is striving into its memory shape. Then the cutting device may be treated in a suitable manner, such as by heat, to “memorize” said memory shape. For example, wires in a web form may be may be formed in accordance with the walls of a mould copy of the tissue to be cut. Then, the cutting device may be formed into its temporary shape, in which the cutting device may be introduced at a suitable site to thereafter perform cutting action in said tissue to be cut, to thereby obtain a homogenous cutting action of the tissue intended to be cut.

The tissue cutting device may thus be shaped specific for a patient's cardiac anatomy. Parameters that may be considered when manufacturing the tissue cutting device are: interior tissue surface, muscle or vessel wall thickness and extent, exterior tissue surface, and adjacent tissue that should be avoided to be cut. For instance a cutting edge providing cutting action of the tissue cutting device may be provided in different sections or progressively changing, depending on the desired degree of cutting action in space or over time, depending on e.g. vessel or heart muscle extent and thickness. Hence patient safety is increased, as for instance damage of surrounding tissue may be avoided. Also, total treatment costs for a patient may be decreased as follow-up surgical procedures are avoided due to the more precise control of cutting action obtained.

Thus, the cutting device 26 may be inserted in this temporary shape to the heart of a patient through the vascular system. The temporary shape of the cutting device 26 is also flexible, whereby guiding the cutting device 26 through the vascular system is facilitated. This insertion of the cutting device 26 may be performed with well-known percutaneous catheter techniques. This is an unaggressive procedure and may be performed on a beating heart. Thus, the cutting device 26 may readily be positioned at a desired position within the vascular system adjacent heart wall tissue to be treated. The cutting device 26 may then be allowed to transfer to its memorized, permanent shape when inserted to the desired position in a blood vessel.

The memorized, permanent shape, which permanent shape has been adapted in respect of the tissue intended to be cut, of the cutting device 26 will not fit into the blood vessel 28, whereby the cutting device 26 will force itself through surrounding tissue for obtaining the permanent shape. In this way, the cutting device 26 will first penetrate the vessel wall and the cutting action of tissue surrounding the blood vessel 28 will be prevented or minimized, since the cutting device has been adapted in respect of the vessel the tissue of which is intended to be cut. Tissue cells that are penetrated will be killed, which will start a healing reaction in the body. Where the cutting device 26 is placed in a desired position to change shape through heart wall tissue, cells that are able to transmit electrical signals may thus be killed. The healing process will not restore the ability to transmit electrical signals and, therefore, the cutting device 26 will reduce the ability of transmitting electrical signals through the heart wall by providing patient configured controllable scarring action.

An example of a shape memory material is Nitinol, which is an alloy composed of nickel (54-60%) and titanium. Small traces of chrome, cobalt, magnesium and iron may also be present. This alloy uses a martensitic phase transition for recovering the permanent shape. Shape memory materials may also be formed of shape memory polymers, wherein the shape-memory effect is based on a glass transition or a melting point. Such shape memory polymers may be produced by forming polymers of materials, or combinations of materials, having suitable properties. For example, a shape memory polymer may be created of oligo(e-caprolactone) dimethacrylate combined with n-butyl acrylate. Also, biodegradable, bio-resorbable, or bioabsorbable materials may be used for forming these shape memory polymers. In this way, the cutting device 26 may be designed such that it will be degraded or absorbed by the body after it has performed its change of shape. For example, a polylactic acid polymer and/or a polyglycolic acid polymer, poly (e-caprolactone) or polydioxanone may be used for forming a shape memory polymer that is biodegradable. A special feature of the resorbable shape memory polymers is that these will disappear from the tissue after having had its function, limiting potential negative effects of otherwise remaining polymer or Nitinol materials, such as perforations and damage to other adjacent tissues, like lungs, esophagus and great vessels like the aorta.

Moreover, other design parameters of tissue cutting devices may be chosen according to patient specific anatomy. Such design parameters are for instance wire thickness distribution, connection points, fastening elements such as hooks, bistable sections or characteristics, material choice, implementation of drug delivery sections, timing design of cutting action, etc. as described in co-pending patent applications concurrently filed by same applicant as present application, which hereby are incorporated by reference herein in their entirety.

The patient configured cutting device 26 may be constructed of a net; i.e. its shape may comprise meshes or loops. This implies that a solid surface need not penetrate tissue, whereby the penetration through tissue and the forming of different shapes of the cutting device 26 will be facilitated. Hereby, the net structure of the cutting device, which cutting device has been adapted in accordance with the patient's vessel or other tissue, which is intended to be cut by the device, will penetrate said tissue in a way that for instance may prevent or regulate the cutting action of a perhaps sensitive tissue surrounding said tissue intended to be cut.

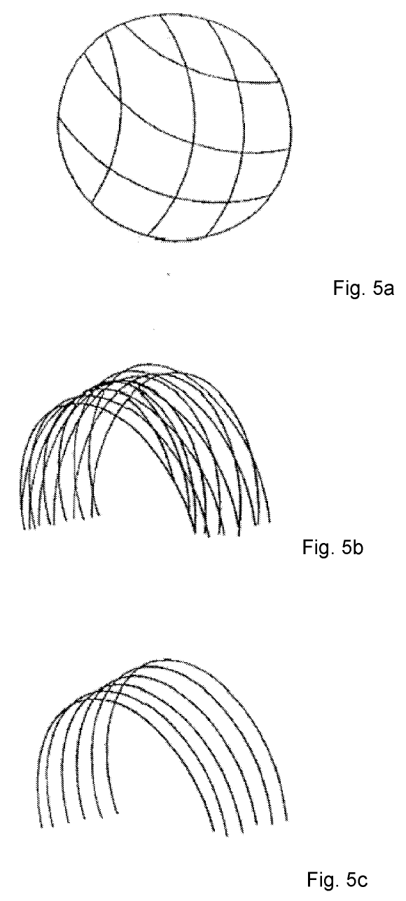

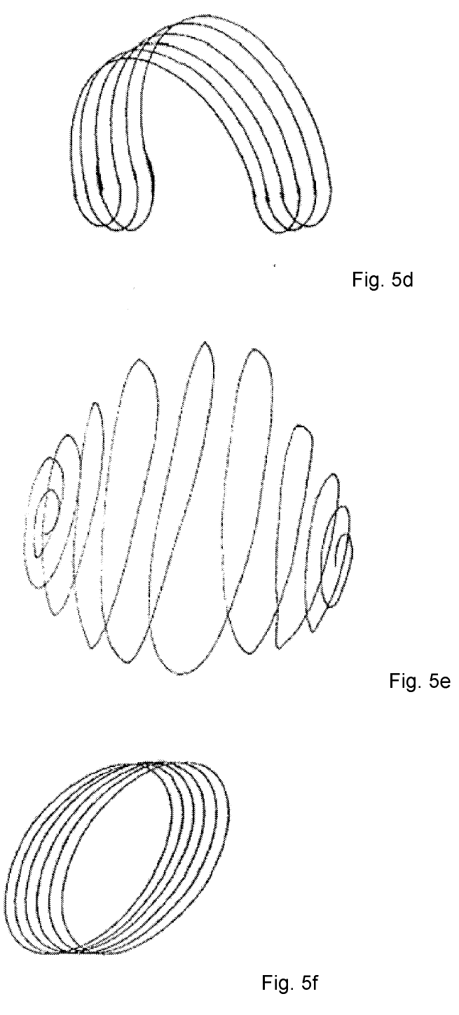

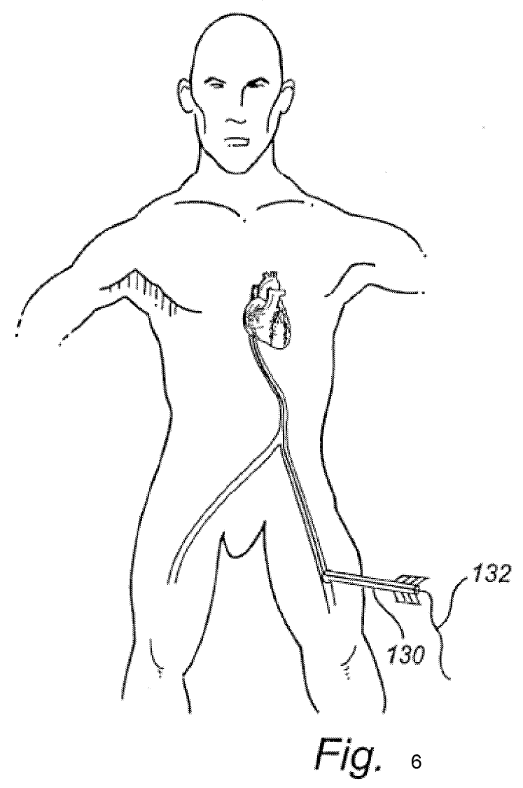

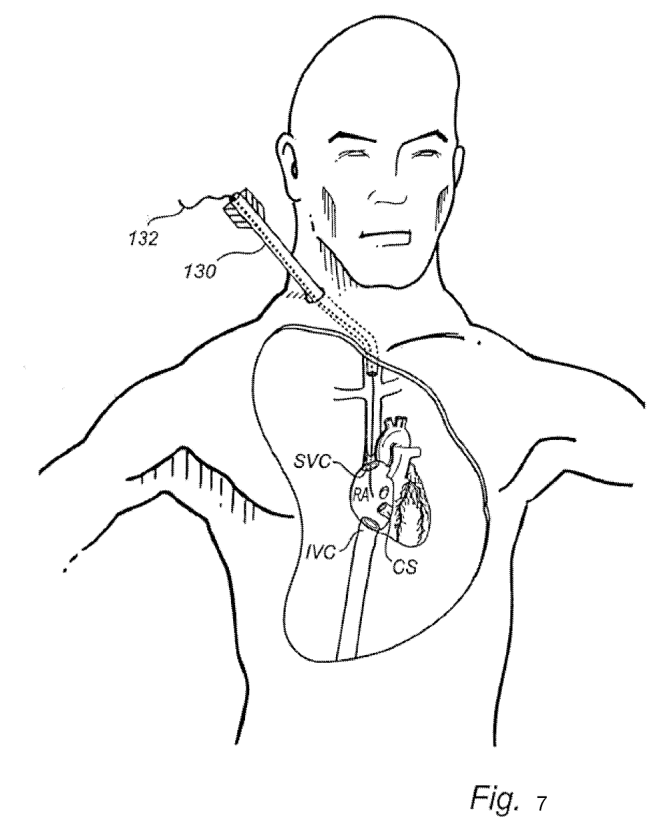

The patient configured cutting device may also comprise one or more cutting arms (not shown), which, in the temporary shape of the cutting device, extend along a tubular part 32 or in an axial direction of the tubular part 32. Further, the cutting device may be arranged to change shape such that the one or more cutting arms extend in a radial direction from the tubular part. Thus, during the change of shape, the one or more cutting arms will penetrate through the tissue intended to be cut. A cutting device according to this embodiment may prevent or minimize cutting of sensitive tissue surrounding the tissue to be cut, while a cutting arm may provided in an arrangement that results in a cutting action, performed by said arm, proceeding past the tissue intended to be cut. This may for example be used when a certain area outside the vessel not is sensitive to cutting while another area is sensitive to cutting. The cutting arm may then provide cutting action in the tissue not sensitive to cutting, while cutting of the tissue sensitive to cutting may be minimized or prevented. This is accomplished since the cutting device is adapted to the actual shape of the vessel, the tissue of which is intended to be cut. It is also possible to interconnect several patient configured tissue cutting devices, e.g. with wires or other connection elements. This embodiment provides for example the advantage of achieving a stabilizing effect of the position of the several cutting devices. One cutting device, which is adapted to the actual shape of a ventricle, may for example be placed in said ventricle, while being interconnected to another cutting device, which is adapted to the actual shape of, and placed in, an atrium. The interconnection wire may then stabilise the respective positions of the cutting device in the ventricle and the cutting device in the atrium. In the embodiment according to FIG. 5a the patient configured cutting device may have a substantially globulus form, to be placed in the atrium or ventricle of a heart. This globulus is placed inside the heart, such as in the left or right atrium, in a temporary shape. The cutting device is then stimulated, by for example temperature, according to above, to expand towards its memorized, permanent shape. This expansion results in that the heart tissue is cut by the cutting device. Tissue cells that are penetrated by the cutting device will be killed, which will start a healing reaction in the body. Where the cutting device is placed in a desired position to change shape through heart wall tissue, cells that are able to transmit electrical signals may thus be killed. The healing process will not restore the ability to transmit electrical signals and, therefore, the cutting device will reduce the ability of transmitting electrical signals through the heart wall. In this case, the globulus shape may be designed patient specific to the patients atrium or ventricle, which comprises that the shape may, at least in a section of the globe, deviate from the globulus shape, e.g. to a pear or bell shaped design in that section.

The patient configured cutting devices according to FIG. 5 may also be combined with the tubular parts of all other embodiments of the present invention, i.e. the cutting devices according to FIG. 9 may be connected with different kinds of tubular parts. These tubular parts may then for example be delivered in a body vessel adjacent the heart while the cutting device according to FIG. 5 is delivered inside the heart.

Now, a system for delivery of a patient configured cutting device into a desired position in a blood vessel adjacent the heart will be described. Each patient configured cutting device may be inserted into its desired position using such a delivery system. Of course, even standard tissue cutting devices off the shelf may be delivered in combination with patient configured tissue cutting devices, depending on the patient or medical requirements on a case to case basis. The delivery system allows a precise placement of each cutting device into the heart and the big vessels of the body. The delivery system has a restraining device, which keeps the cutting device in its temporary shape. This allows insertion into the blood vessel through catheters having a small bore, making minimal trauma to the patient. The restraining device may be a restraining tube, into which the cutting device is forced in its temporary shape. By cooling the cutting device, in case of a cutting device made of Nitinol, it may be easier to force the cutting device into the restraining tube. Once inserted into the desired position, the cutting device may be pushed out of the restraining tube by means of a piston or the cutting device may be released by retracting the restraining tube from its position over the cutting device. In case of a cutting device made of Nitinol, the cutting device may also be restrained by cooling to prevent it from obtaining a transition temperature trigging the change of shape. Thus, the cutting device may be restrained by cooling during insertion into the desired position and released by suspension of the cooling when inserted at the desired position. In WO 03/022179, such a delivery system is described in more detail.

Now, a method for treating a patient having a disorder to the heart rhythm regulation system will be described. The patient is prepared for operation and operation is performed in an environment allowing visualization of the heart and the attached big vessels using fluoroscopy and ultrasound according to conventional techniques.

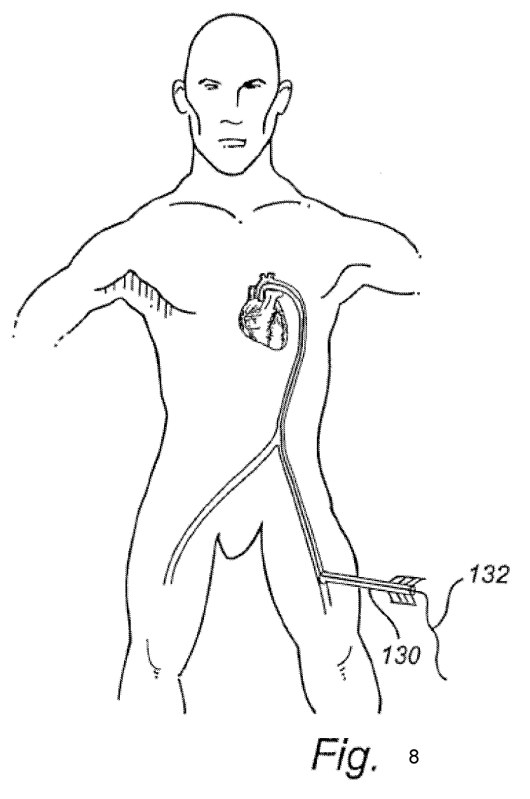

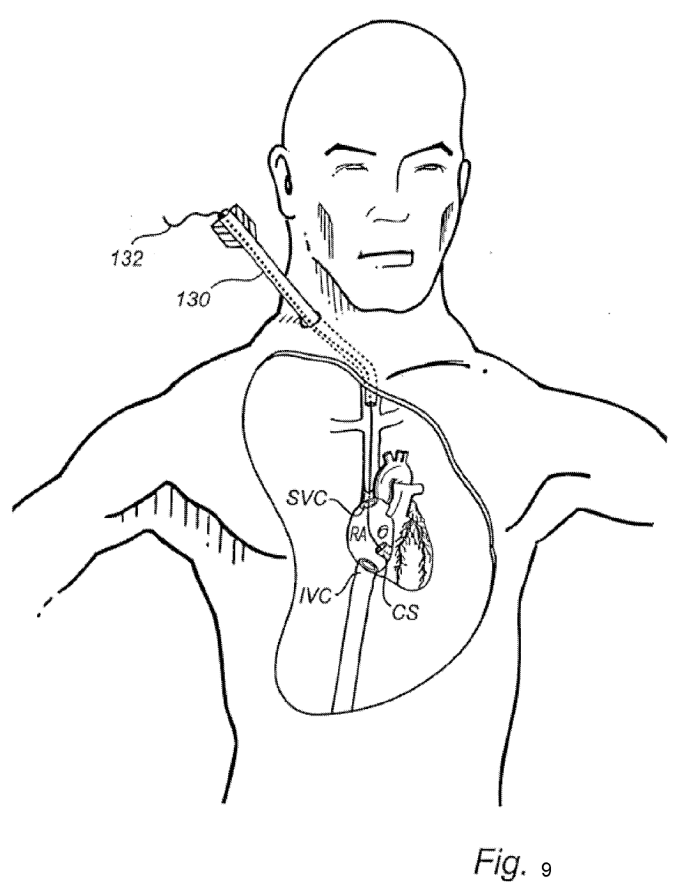

The operation is started by making a puncture of a vein providing an access point to the vascular system of the patient according to conventional techniques. Usually, the femoral vein in the groin, as illustrated in FIG. 6, the subclavian vein on the chest, or the internal or external jugular vein on the neck, as illustrated in FIG. 7, is used. However, other smaller veins may be used instead. Also, in difficult cases when the pulmonary veins cannot be accessed from the vein, arterial access through the femoral artery in the groin may be used, as illustrated in FIG. 8. This method will, however, not be further discussed here. A delivery system is used for inserting the above described cutting devices into blood vessels adjacent the heart. First, an introducer sheath 130 of the delivery system is inserted at the puncture providing an access route into the vascular system. Then, a diagnostic catheter of the delivery system is inserted through the introducer sheath 130 into the vascular system. The diagnostic catheter is manoeuvred through the vascular system into the CS. Next, a guide wire 132 of the delivery system is inserted through a channel of the diagnostic catheter into the CS and all the way to the vein parallel to the left anterior descending artery of the heart, close to the apex of the heart. The guide wire 132 is inserted as far as possible into the vascular system to be firmly positioned. Thereafter, the diagnostic catheter is withdrawn from the patient. The guide wire 132 will then extend from outside the patient into the patient via the access point and inside the patient to the CS, as illustrated in FIG. 9.

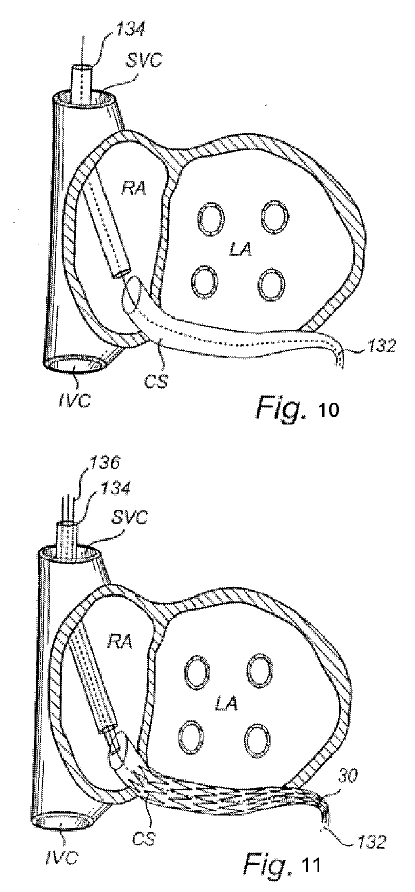

A guide catheter 134 of the delivery system is now inserted over the guide wire 132 so that the guide catheter 134 is positioned with its tip at the orifice of the CS, as illustrated in FIG. 10. Now, there is a guide wire 132 extending from the outside of the patient and the guide catheter 134, through the guide catheter 134, through the CS, the great cardiac vein and the anterior vein parallel to the LAD all the way to the apex of the heart.

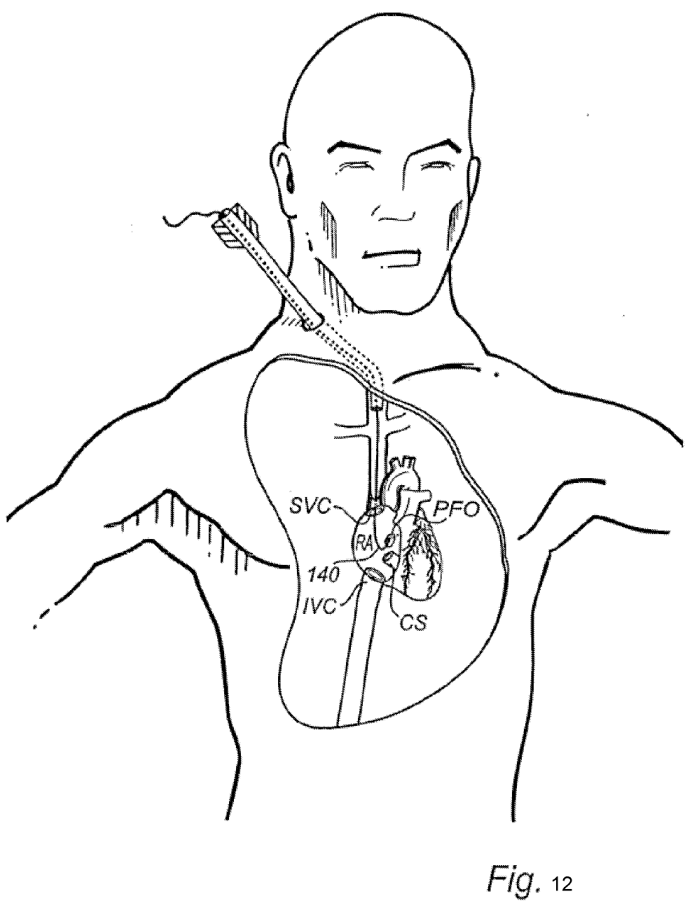

Referring to FIG. 11, a delivery catheter 136 of the delivery system for carrying the first cutting device 30 into the desired position has a guide wire channel throughout its length. The end of the guide wire 132 outside the patient is then inserted into the guide wire channel of the delivery catheter 136, whereby the delivery catheter 136 may be inserted over the guide wire 132 and inside the guide catheter 134 into the CS. The delivery catheter 136 has an inner part providing the guide wire channel and carrying the cutting device at a distal portion. The delivery catheter 136 may further comprise an outer, restraining part, which covers the cutting device and keeps it in a contracted, temporary state. The restraining part may be axially displaceable in relation to the inner part. Thus, the restraining part may be retracted for releasing the cutting device. In this way, the first cutting device 30 is inserted into the CS and may be located in its desired position. A correct position is when the distal end 34 of the first cutting device 30 is positioned within the CS beyond the LIPV next to the CS and the proximal end 36 of the first cutting device 30 is closer to the orifice of the CS than the RIPV. Preferably, the first cutting device 30 extends all the way to the orifice of the CS. Without moving the first cutting device 30 away from its correct position, the first cutting device 30 is released from the delivery catheter. The first cutting device 30 will then immediately expand radially until contact is established with the CS wall, as illustrated in FIG. 11. Thereafter, the delivery catheter 136 is withdrawn from the patient.

However, the first cutting device 30 is arranged to change shape to assume a shape having much larger diameter than the natural diameter of the CS. Thus, the first cutting device 30 will expand to its designed, permanent shape and the CS wall will not be able to prevent the first cutting device 30 from obtaining its permanent shape. In order to obtain its permanent shape, the first cutting device 30 will therefore penetrate tissue in the path of the change of shape. In this way, the first cutting device 30 will expand to penetrate the heart tissue outside the CS, for instance the left atrium wall. The penetrated tissue will be killed and replaced by fibrous tissue, which is not able to transmit electrical signals. Thus, a block against propagation of undesired electrical signals may be created in this manner.

As an option, the first cutting device 30 may be inserted into the CS in a first separate session of the treatment of a patient. Thus, this first cutting device 30 may be allowed to be well-anchored in the tissue around the CS, before other cutting devices are inserted. This is suitable since some of the other cutting devices are adapted to contact the first cutting device 30 inserted into the CS in order to stabilize and fix their positions. The first cutting device 30 will be well-anchored within a few weeks, typically within three weeks. In this time the first cutting device 30 has penetrated the tissue around the CS and is firmly embedded by the tissue fixing its position. Then, the patient will come back for a second session of the treatment. Thus, a puncture is again made into a vein for allowing access again to the vascular system. However, all the cutting devices may alternatively be inserted during one session.

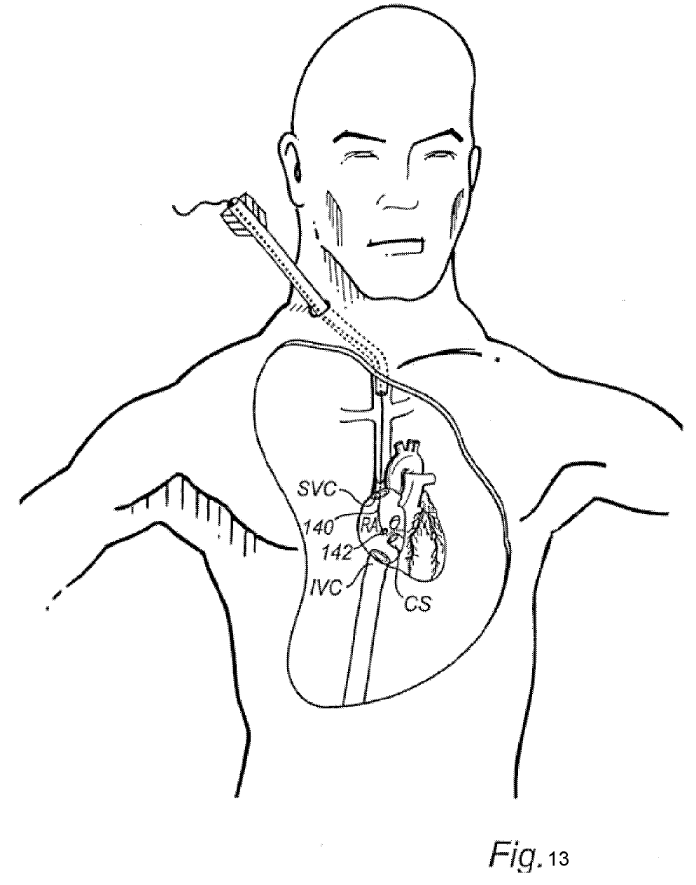

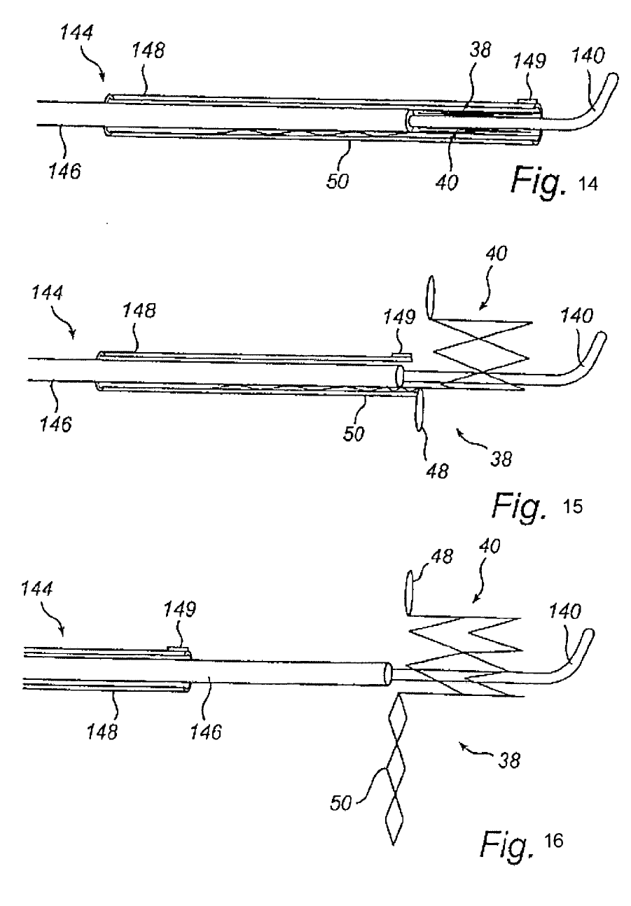

Now, a guide wire 140 is advanced inside a diagnostic catheter into the left atrium (LA), as illustrated in FIGS. 12 and 13. In order to access the LA, the atrial septum between the LA and the right atrium (RA) must be penetrated. If the patient has a patent foramen ovale (PFO, FIG. 12), which is an opening between the LA and the RA that is normally only present during the fetal period in humans, this may be used and enlarged, for instance by means of a balloon catheter (not shown). If no PFO is present (FIG. 13), a small opening 142 must first be created by means of a long flexible needle passed through a diagnostic catheter inside the access vein. Again, the opening 142 in the atrial septum may be enlarged by means of a balloon. Once the needle is inside the LA, the catheter is passed over the needle into the LA and the needle is retracted. A guide wire 140 may now be advanced through the catheter into the LA and further into the LIPV.

Referring now to FIGS. 14-16, the release of a cutting device will be generally described. Thus, having now placed the guide wire 140, the second cutting device 38 may be inserted to its desired position using a guide catheter extending to the LIPV orifice and a delivery catheter 144, as illustrated in FIG. 14, in a similar manner as for the insertion of the first cutting device 30. The delivery catheter 144 has an inner part 146 providing the guide wire channel. The tubular part 40 of the second cutting device 38 is arranged in front of the inner part 146 such that the inner part 146 of the delivery catheter 144 pushes the tubular part 40 in front of it. The delivery catheter 144 may further comprise an outer, restraining part 148, which covers the cutting device and keeps it in a contracted, temporary state. The restraining part 148 may be axially displaceable in relation to the inner part 146. Thus, the restraining part 148 may be retracted for releasing the cutting device 38. The delivery catheter 144 has a marker on the catheter outside the patient, as well as a x-ray marker 149 visible on the fluoroscopy, indicating securely the orientation of the cutting arm 50 of the second cutting device 38. The second cutting device 38 is now rotated into a position where it will change shape in such a way that the cutting arm 50 will extend to contact and be supported by the first cutting device 30, which has been inserted previously. The second cutting device 38 is advanced into a position where the atrial end 48 of the second cutting device 38 is still outside the LIPV orifice. When the correct position of the second cutting device 38 is confirmed by means of fluoroscopy and/or ultrasound, the distal end of the second cutting device 38 is released from the delivery catheter far inside the PV, whereby the distal end will expand radially to fix the position of the second cutting device 38. Next, a mid portion of the second cutting device 38 and the atrial end 48 is released, as illustrated in FIG. 15. Now, the cutting arm 50 is released, as illustrated in FIG. 16, and allowed to assume its radial extension from the tubular part 40, whereby it will penetrate the heart wall to contact the first cutting device 30.

Now, the guide wire 140 is retracted into the LA. The diagnostic catheter is inserted again and guided into the RIPV, whereby the guide wire 140 may be inserted into the RIPV. Thereafter, the diagnostic catheter is withdrawn from the patient. Then, the third cutting device 54 is inserted using a guide catheter extending to the RIPV orifice and a delivery catheter 144 in a manner similar to the insertion of the second cutting device 38. Thus, the orientation of the cutting arm 66 of the third cutting device 54 is determined in the same manner as for the second cutting device 38. Having correctly positioned the third cutting device 54, the tubular part 56, the atrial end 64 and the cutting arm 66 of the third cutting device 54 are released in a manner similar to the release of the second cutting device 38. Now, the cutting arm 66 is released and allowed to assume its radial extension from the tubular part 56, whereby it will penetrate the heart wall to contact the first cutting device 30.

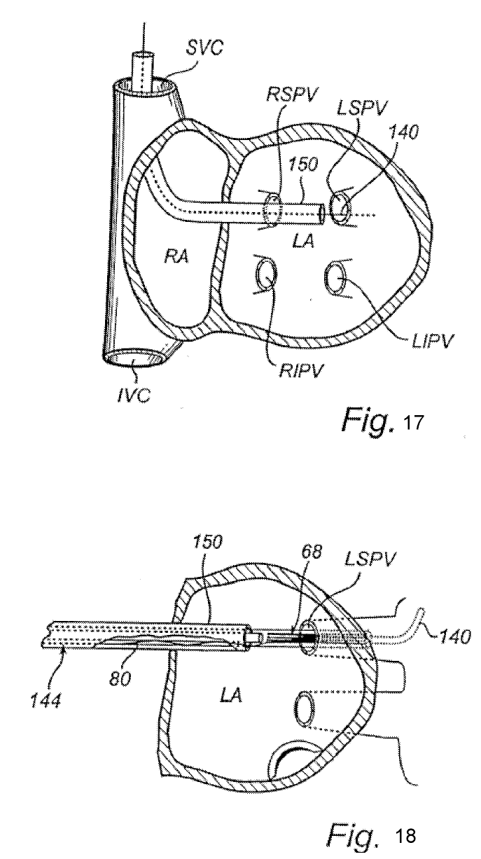

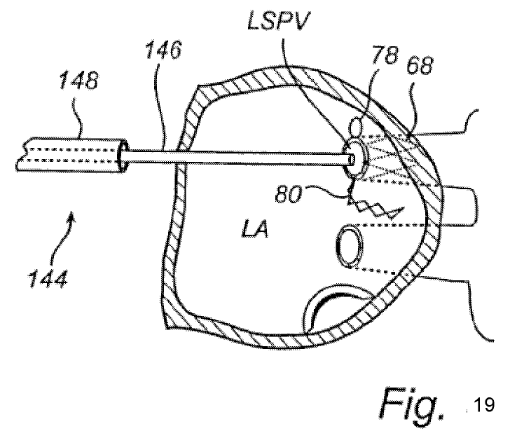

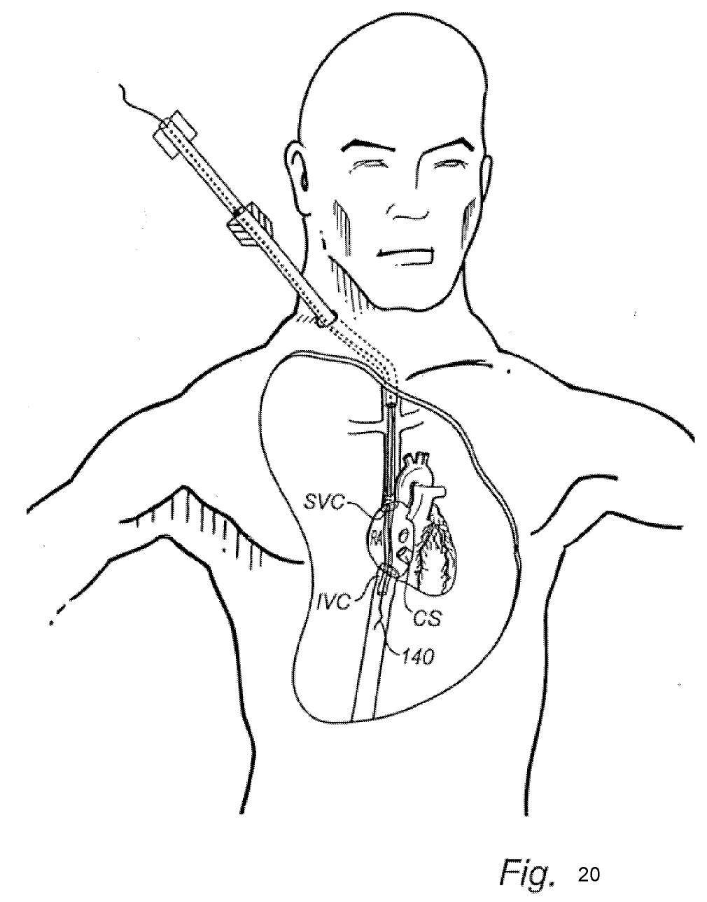

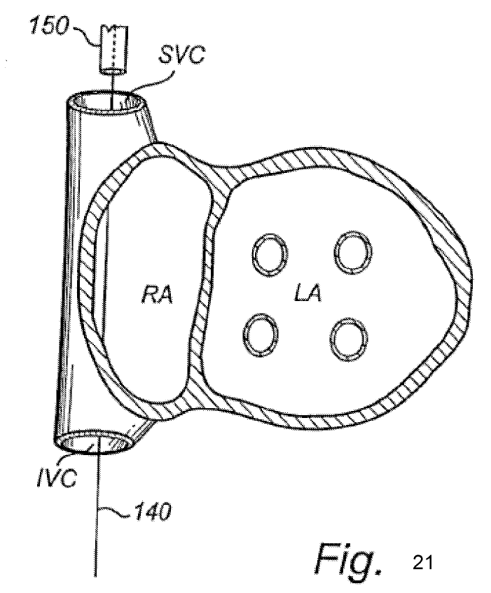

Thereafter, the guide wire 140 is again retracted into the LA and inserted into the LSPV, as illustrated in FIG. 17. Then, the fourth cutting device 68 is inserted using a guide catheter 150 extending to the LSPV orifice and a delivery catheter 144, as illustrated in FIG. 18, in a manner similar to the insertion of the second and third cutting devices 38, 54. Thus, the orientation of the cutting arm 80 of the fourth cutting device 68 is determined in the same manner as for the second and third cutting devices 38, 54. The fourth cutting device 68 may have two cutting arms, which are adapted to extend towards the second cutting device 38 and towards the LAA. Having correctly positioned the fourth cutting device 68, the tubular part 70, the atrial end 78 and the one or two cutting arms 80 of the fourth cutting device 68 are released in a manner similar to the release of the second and third cutting devices 38, 54, as further illustrated in FIG. 19. Now, the cutting arms are released and allowed to assume their radial extension from the tubular part 70, whereby they will penetrate the heart wall to contact the second cutting device 38 or extend to the orifice of the LAA, respectively.

Again, the guide wire 140 is retracted into the LA and inserted into the RSPV. Then, the fifth cutting device 82 is inserted using a guide catheter 150 extending to the RSPV orifice and a delivery catheter 144 in a manner similar to the insertion of the second, third and fourth cutting devices 38, 54, 68. Usually, the fifth cutting device 82 has no cutting arm and therefore only the axial position of the fifth cutting device 82 needs to be determined. Having correctly positioned the fifth cutting device 82, the tubular part 84, and the atrial end 92 of the fifth cutting device 82 are released in a manner similar to the release of the second, third, and fourth cutting devices 38, 54, 68.

Once again, the guide wire 140 is retracted into the LA and now inserted into the LAA. Then, the sixth cutting device 94 is inserted using a guide catheter 150 extending to the LAA orifice and a delivery catheter 144 in a manner similar to the insertion of the other cutting devices. The sixth cutting device 94 is advanced into a position where the entire sixth cutting device 94 is inside the LAA, and a proximal end of the sixth cutting device 94 is adjacent to the LAA orifice. The delivery catheter 144 has a marker on the catheter outside the patient, as well as a x-ray marker 149 visible on the fluoroscopy, indicating securely the orientation of the sixth cutting device 94 such that the elliptic shape of the sixth cutting device 94 may be oriented in correspondence to the elliptic shape of the LAA. When the correct position of the sixth cutting device 94 is confirmed by means of fluoroscopy, a distal end of the sixth cutting device 94 is released from the delivery system far inside the LAA, whereby the distal end will expand radially towards the wall of the LAA to fix the position of the sixth cutting device 94. Next, a mid portion of the sixth cutting device 94 and a proximal end are released. Now, the sixth cutting device 94 is allowed to change its shape to cut through the heart wall of the LAA.

Now, the guide wire 140 is retracted from the LA into the RA and inserted into the RAA. Then, another sixth cutting device 94 is inserted using a guide catheter 150 extending to the RAA orifice and a delivery catheter 144 in a manner similar to the insertion of the other cutting devices. The other sixth cutting device 94 is advanced into a position where the entire sixth cutting device 94 is inside the RAA, and a proximal end of the sixth cutting device 94 is adjacent to the RAA orifice. The position of the sixth cutting device 94 is determined in a manner similar to the positioning of the sixth cutting device 94 inserted into the LAA. When the correct position of the sixth cutting device 94 is confirmed, the sixth cutting device 94 inserted into the RAA is released in a manner similar to the release of the sixth cutting device 94 inserted into the LAA. Now, the sixth cutting device 94 is allowed to change its shape to cut through the heart wall of the RAA.

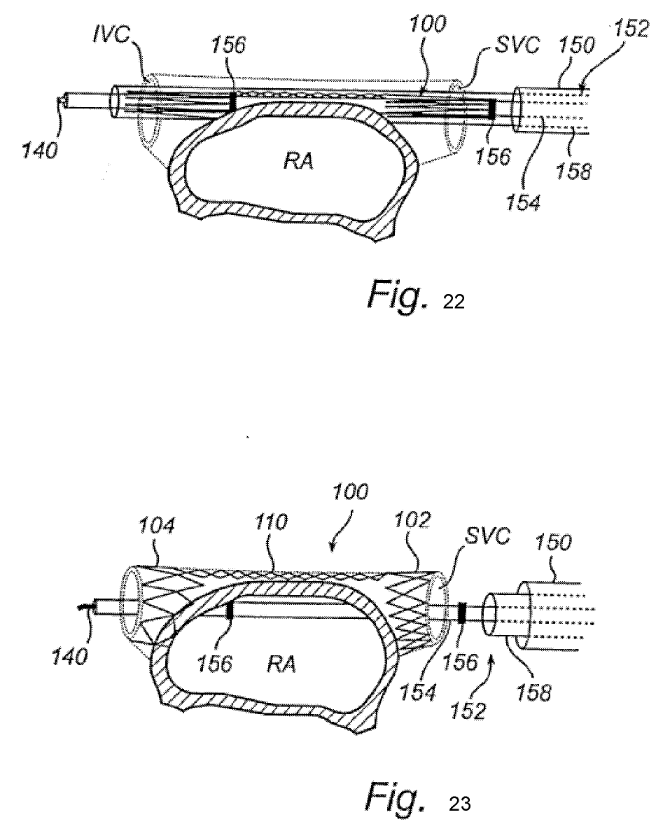

Next, the guide wire 140 is retracted from the RAA into the RA. If the access point to the vascular system was created in the upper part of the body, the guide wire 140 extends through the SVC into the RA. Then, the guide wire 140 is further inserted into the IVC, as illustrated in FIG. 20. On the other hand, if the access point to the vascular system was created in the lower part of the body, the guide wire 140 extends through the IVC into the RA. Then, the guide wire 140 is further inserted into the SVC. Thereafter, the seventh cutting device 100 is inserted using a guide catheter 150, as illustrated in FIG. 21, and a delivery catheter 144 in a manner similar to the insertion of the other cutting devices. The seventh cutting device 100 is placed in position in the IVC, SVC and the RA, as illustrated in FIG. 22. The delivery catheter 152 carries the seventh cutting device 100 on the inner part 154 of the catheter 152. The inner part 154 comprises stops 156, which prevent the seventh cutting device 100 from being axially displaced from the inner part 154 during insertion of the device. Again, the cutting device 100 is kept in a contracted, temporary state by means of a restraining part 158. The correct orientation of the seventh cutting device 100 is obtained in a manner similar to the positioning of the second, third and fourth cutting devices 38, 54, 68. The seventh cutting device 100 has now been rotated into a position where it will change shape in such a way that its cutting arm or cutting arms 122 will extend in intended directions. Thus, the seventh cutting device 100 may comprise a cutting arm 122 that extends towards the orifice of the CS and/or a branch 112 that extends from the connecting cutting arm 110 of the seventh cutting device 100 towards the lateral wall of the RA. When the correct position of the seventh cutting device 100 is confirmed by means of fluoroscopy, a distal end of the seventh cutting device 100 in the delivery catheter 152 is released from the delivery catheter 152 in the IVC or SVC, depending on where the distal end of the delivery catheter is placed. Thereafter, the connecting cutting arm 110 is released and finally a proximal end of the seventh cutting device 100 is released, as illustrated in FIG. 23.

Now, the guide wire 140 and the delivery catheter 152 is retracted outside the patient, since all parts of the treatment kit have been implanted.

On special indication, for instance when it is difficult to place the guide wire inside the PVs, an arterial access may be used instead. The insertion technique is identical, except that the access to the vascular system is achieved by puncture of an artery and that the cutting devices are delivered through the arterial system instead of through the venous system. After puncture of the artery, a catheter is advanced through the aorta and passed by the aortic valve into the left ventricle and finally into the LA. The guide wire is advanced into the desired PV and the insertion of the cutting device may then be achieved in the manner described above.

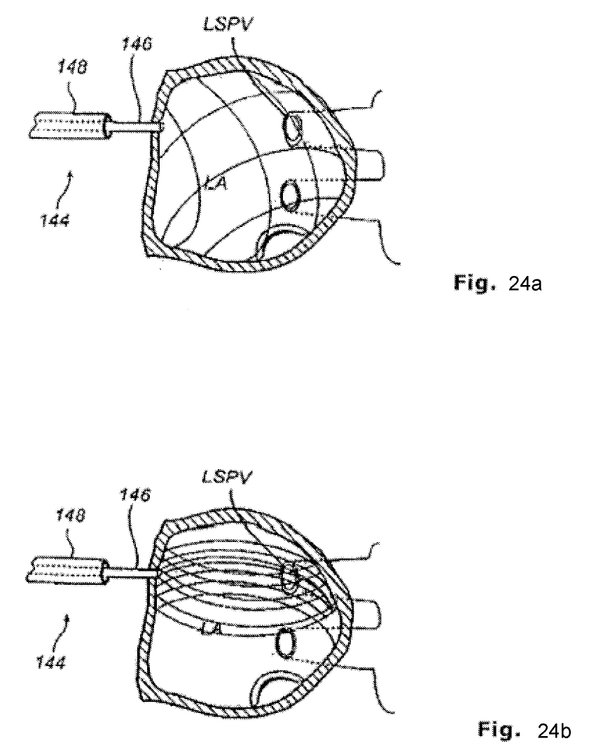

Referring now to FIGS. 24a and b, the release of a cutting device, according to FIG. 5, into the left atrium will be generally described. Thus, having now placed the guide wire 140, the cutting device according to FIG. 5 may be inserted to its desired position using a guide catheter extending to the LA and a delivery catheter 114, as illustrated in FIG. 14, in a similar manner as for the insertion of the first cutting device 30. The delivery catheter 144 has an inner part 146 providing the guide wire channel. The guiding catheter and the delivery catheter are advanced well into the LA so that when releasing the device into the LA the device gets contact with the wall furthest away, the guiding catheter is retracted into the RA and the restraining catheter is retracted towards the atrial septum causing the device to be released into the LA. The catheters and the guide wire are retracted to outside the patient.

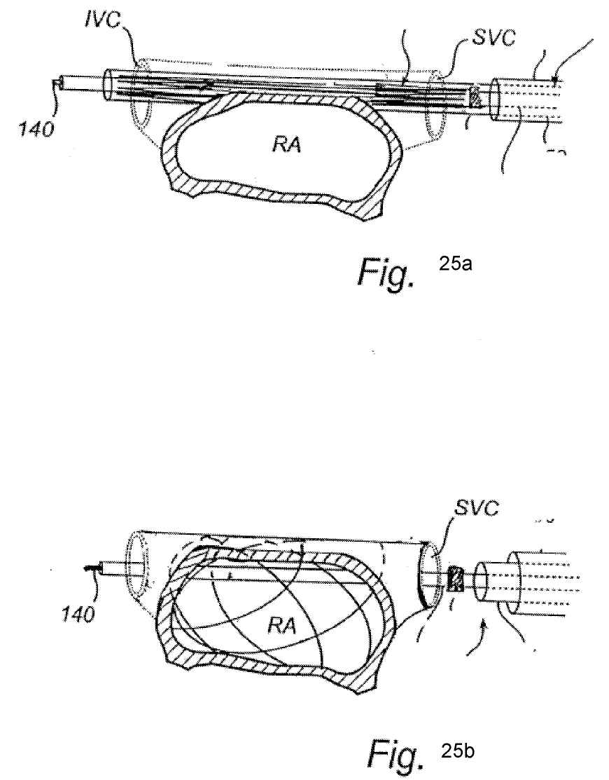

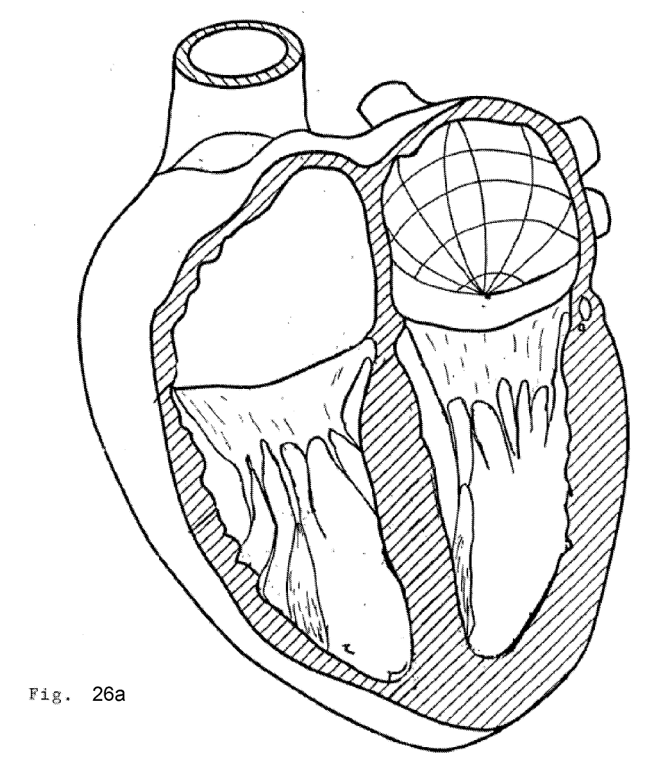

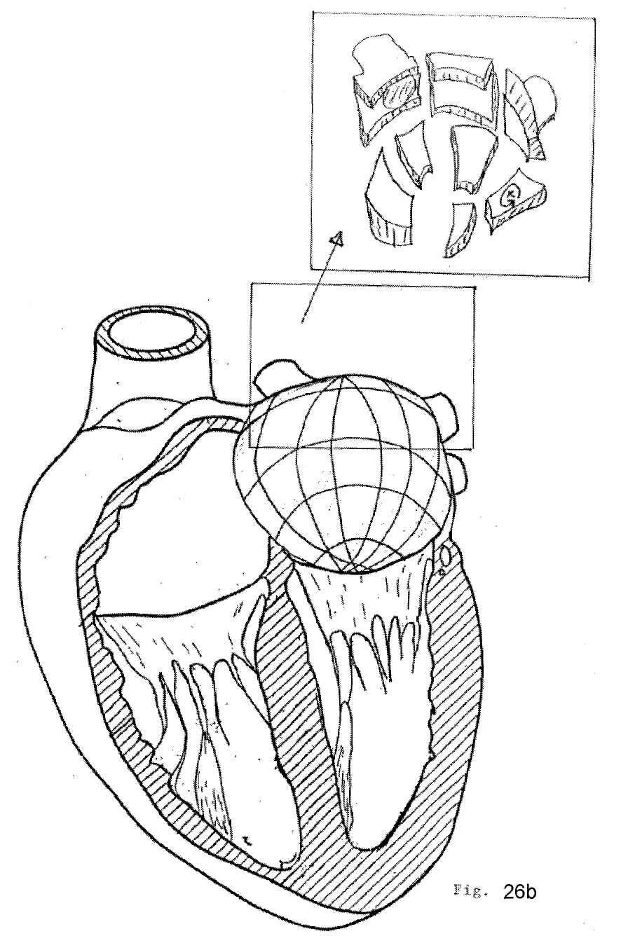

Now a release of the device in the RA is described. The guide wire is advanced into the IVC if the approach is from the neck and into the SVC if the approach is from the groin, according to FIGS. 25a and b. The delivery catheter is advanced to the most distant point where the atrial device is to be deploid, the restraining catheter is retracted towards the SVC or IVC respectively, causing the device to be released into the RA, according to FIG. 25b. The catheters and the guide wire are retracted to outside the patient.

FIG. 26a shows the cutting device according to FIG. 9a positioned in the RA, and FIG. 26b shows the same cutting device in the permanent, expanded shape, i.e. when the wall of the RA has been cut.

The cutting devices according to the present invention have now been released such that they may change their shapes to obtain their permanent shapes. During the change of shape, each cutting device will penetrate heart tissue in the path of the change of shape. Thus, the cutting devices will now create the cutting pattern intended for forming blocks against propagation of undesired electrical signals in the heart. After the cutting devices have made their change of shape, the needed effect of the cutting devices on the heart tissue is completed. Thus, if the cutting devices are made of resorbable shape memory polymers, the cutting devices will be resorbed a time after termination of the cutting procedure. This time for resorption can be set by determination of the different ingredients of polymers and also by means of external altering, for instance by means of x-ray radiation, ultrasound, electron beams, or light of a defined wavelength, setting the time of the polymers to be resorbed. However, the cutting devices may also be left in the body after the change of shape, or only some of the cutting devices may be resorbed.

Moreover, other design parameters of tissue cutting devices may be chosen according to patient specific anatomy. Such design parameters are for instance wire thickness distribution, connection points, fastening elements such as hooks, bistable sections or characteristics, material choice, implementation of drug delivery sections, timing design of cutting action, etc. as described in co-pending patent applications concurrently filed by same applicant as present application, which hereby are incorporated by reference herein in their entirety.

Hereinafter, some potential uses of the present invention are described:

A method for treatment of disorders in the heart rhythm regulation system, said method comprising:

inserting a tissue cutting device through the vascular system to a desired position in a body vessel, and providing a change of shape of the tissue cutting device at said desired position to penetrate heart tissue adjacent said body vessel.

The method according to above, wherein said tissue cutting device is inserted into a desired position in the coronary sinus, in any of the pulmonary veins, in the superior vena cava, in the inferior vena cava, or in the left or right atrial appendage.

The method according to above, further comprising inserting another tissue cutting device to another of the desired positions.

The method according to above, further comprising inserting a tissue cutting device into each of the desired positions.

The method according to above, further comprising restraining the tissue cutting device in an insertion shape during the inserting of the tissue cutting device.

The method according to above, wherein the restraining comprises keeping the tissue cutting device inside a tube.

The method according to above, wherein the restraining comprises cooling the tissue cutting device.

The method according to above, further comprising releasing a restrain on the tissue cutting device when it has been inserted into the desired position for allowing said change of the shape of the tissue cutting device.

Herein above, specific embodiments of the invention have been described with reference to the drawings. However, the invention may be varied within the embodiments shown. The different separate features may be combined in other combinations than specifically disclosed. The invention is only limited by the appended patent claims.

1-39. (canceled)

40. A method of manufacturing at least one tissue cutting device configured for reducing undesired signal transmission in a cardiac tissue of a patient, comprising
non-invasively creating a medical patient image of a cardiac tissue region of said patient, said medical image comprising information of said cardiac tissue intended to be cut by said tissue cutting device, wherein said cardiac tissue region comprises at least one blood vessel, having at least one vessel tissue wall intended to be cut by said manufactured tissue cutting device;
deriving parameters for manufacturing of said tissue cutting device including interior tissue surface, muscle or vessel wall thickness and extent, exterior tissue surface, and adjacent tissue that shall be avoided to be cut from said medical patient image; and
forming said tissue cutting device as a patient configured tissue cutting device in respect of said medical patient image; wherein said forming comprises providing a cutting edge on said tissue cutting device providing cutting action of said tissue cutting device in different sections or progressively changing, depending on said desired degree of cutting action in space or over time in said tissue, and
providing said cutting device with a memorized, permanent shape, and adapting said permanent shape in respect of said vessel tissue wall such that said cutting device does not fit into said blood vessel, such that said cutting device, when inserted to a desired position in said blood vessel, is provided to force itself through surrounding tissue for obtaining said permanent shape by first penetrating said vessel wall, whereby said cutting device is adapted to reduce said ability of transmitting electrical signals through said vessel tissue by providing patient configured controllable scarring action.

41. The method according to claim 40, wherein said method comprising capturing a heart cycle movement by means of a series of said medical patient images, and said method comprising dynamically adapting said tissue cutting device to specific patient anatomy for said patient configured controllable scarring action.

42. The method according to claim 40, comprising providing a wire thickness distribution, connection points, fastening elements such as hooks, bistable sections or characteristics, material choice, drug delivery or release sections, or timing design of cutting action of said tissue cutting device for at least partly conforming with said vessel tissue of said patient.

43. The method according to claim 40, wherein said medical image is a three dimensional (3-D) image of said cardiac tissue region providing a three dimensional model thereof.

44. The method according to claim 40, comprising forming a copy of said cardiac tissue region from said medical image, which copy is used as a template when forming said patient configured tissue cutting device in respect of said medical image.

45. The method according to claim 44, wherein said copy is made by means of 3-D rapid manufacturing (RM), such as layer manufacturing or Solid Freeform Fabrication (SFF).

46. The method according to claim 45, wherein said copy is made in a larger dimension than said actual image, whereby said cutting device is formed into said memory shape.

47. The method according to claim 46, further comprising treating said cutting device to memorize said memory shape, wherein said treatment is a heat treatment.

48. The method according to claim 40, further comprising forming said cutting device into a temporary delivery shape.

49. The method according to claim 40, wherein said non-invasively creating of an image is performed by Magnetic Resonance Tomography (MRT), Magnetic Resonance Imaging (MRI), Positron Emission Tomography (PET), CT or CAT scans, or Ultrasonic imaging systems.

50. The method according to claim 40, wherein said tissue cutting device is patient configured for reducing undesired signal transmission in a said cardiac tissue of said patient by isolating ectopic sites thereof by said cutting of said tissue,
said method comprising structuring and arranging said cutting device to be inserted in a temporary delivery shape through said vascular system into a body vessel adjacent to said heart and/or into said heart of said patient and to be subsequently subjected to a change of shape, from said temporary delivery shape via an expanded delivered shape to a further expanded shape, in order to create said cutting action configured for cutting said cardiac tissue and/or said blood vessel, and wherein said cutting device has a shape adapted to said actual shape of said body vessel adjacent to said heart and/or said heart of said patient.

51. The method according to claim 40, wherein said forming comprises forming said tissue cutting device of a biodegradable, bioresorbable, or bioabsorbable material and/or a shape memory material.

52. The method according to claim 40, wherein said forming comprises forming said cutting device to change shape to expand its dimensions in a radially outward oriented direction thereof, to thereby provide said cutting action for cutting said blood vessel.

53. The method according to claim 40, wherein said forming comprises forming said cutting device in a substantially globular shape.

54. The method according to claim 40, wherein said tissue cutting device is patient configured for reducing undesired signal transmission in a said cardiac tissue by isolating ectopic sites thereof by said cutting action for cutting said tissue,
said method comprising structuring and arranging said cutting device to be inserted in a temporary delivery shape through said vascular system into a body vessel adjacent to said heart, and to be subsequently subjected to a change of shape, from said temporary delivery shape via an expanded delivered shape to a further expanded shape, in order to create said cutting action configured for cutting said cardiac tissue and/or said body vessel, wherein said method comprises
forming said cutting device of a plurality of segments connected to each other in a longitudinal direction of said cutting device, wherein a first segment of said plurality of segments is given a dimension in a direction perpendicular to said longitudinal direction of said device larger than a dimension of a second segment thereof, at least in said expanded delivered shape, and said forming comprises giving said cutting device a shape adapted to said actual shape of said body vessel adjacent to said heart and/or said heart, wherein said second segment is formed to be configured to fit in a branch of said pulmonary vein system having a smaller diameter than said first segment, and said method further comprising providing said cutting device with at least one cutting arm being structured and arranged to initially extend substantially perpendicular to said longitudinal direction from said tissue cutting device in order to be inserted into a heart atrium wall and said cutting arm being structured and arranged to change shape to extend radially from said tissue cutting device, or said body vessel, which said device is structured and arranged to be inserted into, is said coronary sinus.

55. The method according to claim 40, said method comprising forming a plurality of said patient configured cutting devices for treatment of disorders in said heart rhythm regulation system by shape-changing of said cutting devices in said patient,
wherein each of said cutting devices of said a plurality of said shape-changing patient configured cutting devices, is formed with a first delivery and a second delivered state, wherein said cutting device in said first state has such dimensions as to be insertable to a desired position within said vascular system at said blood vessel, and wherein said device formed to be capable of changing shape to substantially said second state when located at said desired position, which strives to a diameter that is larger than said diameter of said vessel at said desired position, whereby said device is formed to become embedded into tissue surrounding said blood vessel at said desired position and destroy said tissue in order to prevent it from transmitting electrical signals,
wherein at least one of said shape-changing devices is adapted to be inserted to a desired position at said orifice of a pulmonary vein in said heart, and at least one of said shape-changing devices is adapted to be inserted to a desired position in said coronary sinus, wherein at least one cutting device has a shape adapted to said actual shape of said body vessel adjacent to said heart and/or said heart; and wherein at least one of said shape-changing devices is adapted to be inserted into said inferior vena cava, or wherein at least one of said shape-changing devices is adapted to be inserted into said superior vena cava.

56. The method according to claim 55, further comprising interconnecting several of said plurality of patient configured cutting devices with connection elements, whereby said several cutting devices are configured to be stabilized at respective positions of said cutting device upon placement in said patient.
